(19) United States
(12) Patent Application Publication
(10) Pub. No.: US 2023/0293765 A1
(43) Pub. Date: Sep. 21, 2023

(54) MEDICAL MATERIAL FOR PROMOTING CELL GROWTH AND INHIBITING BACTERIAL ADHESION AND MACHINING METHOD THEREOF

(71) Applicant: HEBEI UNIVERSITY OF TECHNOLOGY, Tianjin (CN)

(72) Inventors: Chunyong LIANG, Tianjin (CN); Xianrui ZOU, Tianjin (CN); Hongshui WANG, Tianjin (CN); Tai YANG, Tianjin (CN); Huan ZHOU, Tianjin (CN)

(21) Appl. No.: 18/324,847

(22) Filed: May 26, 2023

Related U.S. Application Data
(63) Continuation of application No. PCT/CN2022/097080, filed on Jun. 6, 2022.

(30) Foreign Application Priority Data
Jun. 17, 2021 (CN) 202110673189.0

(51) Int. Cl. A61L 27/32; A61L 27/30

(52) U.S. Cl. CPC A61L 27/32; A61L 27/306; A61L 2300/404; A61L 2400/12; A61L 2420/02; A61L 2420/06; A61L 2430/02

(57) ABSTRACT

Provided are a medical material for promoting cell growth and inhibiting bacterial adhesion and a machining method thereof. The machining method comprises: modifying a surface component of the medical material; preparing a micro-nano structure formed by superposing multiple levels of sizes; and selecting one of the two steps above, or carrying out component modification on a surface of the medical material first and then forming the micro-nano structure by superposing the multiple levels of sizes. The micro-nano structure formed by superposing the multiple levels of sizes comprises a first-level structure which is a micron-level groove structure, a second-level structure which is a submicron-level stripe structure or an array protrusion structure and a third-level structure which is a nano-level protrusion structure, the second-level structure is distributed on a surface of the first-level structure, and the third-level structure is distributed on a surface of the second-level structure.

### CROSS-REFERENCE TO RELATED APPLICATIONS

This application is a continuation of International Patent Application No. PCT/CN2022/097080 with a filing date of Jun. 6, 2022, designating the United States, now pending, and further claims priority to Chinese Patent Application No. 202110673189.0 with a filing date of Jun. 17, 2021. The content of the aforementioned applications, including any intervening amendments thereto, are incorporated herein by reference.

### TECHNICAL FIELD

The present invention belongs to the field of medical materials, and relates to a material modification technology and a laser machining technology, and particularly to a medical material for promoting cell growth and inhibiting bacterial adhesion and a machining method thereof.

### BACKGROUD OF THE PRESENT INVENTION

Biomedical materials are used for diagnosing, treating, repairing or replacing impaired tissue and organ of a living body or improving functions of the impaired tissue and organ. All kinds of implanted and intervened medical devices prepared by using biomedical metals, bioceramics and polymer biomaterials have been widely used in many fields such as orthopedics, dentistry, skin and tendon repair, cardiovascular disease processing, and cancer processing.

With the deepening of clinical practice, more attention has been paid to endowing surfaces of the medical materials with functions of promoting tissue integration and resisting bacterial infection. Because most of the medical materials currently used in clinic are bio-inert materials, there is a lack of promoting cell adhesion and growth and tissue integration, so that the rapid integration of the materials with surrounding tissues cannot be realized, thus having a poor curative effect. Meanwhile, there is also a risk of bacterial infection on the surfaces of the materials, which may lead to an inflammatory reaction of an implanting part and surgical failure, and even endanger lives of patients in serious cases. Bacteria exist in every corner of daily life, and there is a risk of bacterial infection in preparing, transportation, storage and use of the medical devices. However, once the bacteria are adhered to surfaces of the devices, it is easy to form a bacterial biofilm in suitable conditions, and colonies after film formation easily cause various bacterial infections. Therefore, there is an urgent clinical demand on how to realize cell growth promotion and bacterial resistance of the medical materials at the same time. Surface modification carried out on the medical materials is an important method to solve the above problem. The surface modification endows the surfaces of the medical materials with specific functions by changing a physical structure or a chemical composition of the surfaces of the medical materials without affecting a performance of a material matrix.

### SUMMARY OF PRESENT INVENTION

The present invention aims to solve the problem that medical materials lack capabilities of promoting cell growth and inhibiting bacterial adhesion, and the present invention provides a surface structure of a medical material with dual functions of promoting cell adhesion and growth and inhibiting bacterial adhesion and proliferation and a machining method thereof. The surface structure is a coating or a plating with dual functions of promoting cell growth and inhibiting bacterial adhesion and proliferation, and meanwhile, the coating or the plating has a micro-nano structure with dual functions of promoting cell adhesion and inhibiting bacterial adhesion.

A technical solution used to realize the present invention is as follows:

a machining method of a surface structure of a medical material for promoting cell growth and inhibiting bacterial adhesion comprises:
modifying a surface of the medical material;
preparing a micro-nano structure formed by superposing multiple levels of sizes; and
selecting one of the two steps above, or carrying out modification on the surface of the medical material first and then forming the micro-nano structure by superposing the multiple levels of sizes.

The modification refers to preparing at least one of a coating and a plating on the surface of the medical material.

The micro-nano structure above may be prepared only on a surface of a matrix of a biomedical material without modifying a surface component, or at least one of a coating and a plating with a specific component may be prepared only on the surface without preparing the micro-nano structure, and effects of promoting cell adhesion and inhibiting bacterial adhesion can also be achieved.

The plating comprises one or two or more elements of Ca, Zn, Fe, Ta, Mo, Ti, Au, Pt, Cu, Ag, P, Se, B, C, N, Ar and He capable of improving wear resistance, corrosion resistance, antibacterial ability and biocompatibility of the material.

The coating comprises one or two or more compounds of hydroxyapatite, TiO₂, SiO₂ and ZrO₂ with good wear resistance, corrosion resistance and biocompatibility.

A thickness of at least one of the coating and the plating is 10 nm to 500 µm, and a height difference between a highest point and a lowest point of the micro-nano structure formed by superposing the multiple levels of sizes is less than the thickness of at least one of the coating and the plating.

At least one of the coating and the plating may be a coating or a plating containing one of the components above, a coating or a plating containing a plurality of the components above, or a combination of different coatings and different platings.

At least one of the coating and the plating is prepared by one method or a combination of two or more methods of plasma injection, plasma sputtering coating, plasma spraying, laser cladding, pulse laser deposition, laser alloying, sol-gel electrochemical deposition, electrophoretic deposition, anodic oxidation or micro-arc oxidation, method, and loaded on the surface of the medical material or added to the surface of the medical material. By controlling a related process for preparing the coating or the plating, a coating or a plating with a specific thickness may be prepared on the surface of medical material, and different functions may be endowed to the surface of the medical material by preparing different coatings or platings, for example, Ca, P, Ta and other elements and hydroxyapatite and other compounds can effectively improve the biocompatibility of the surface of the medical material and promote cell adhesion and growth, and Zn, Cu, TiO₂ and other components can effectively inhibit bacterial adhesion and proliferation.

In order to make the surface of the medical material promote cell adhesion and growth and inhibit bacterial adhesion and proliferation more effectively, and ensure long-term effectiveness of cell promotion and bacterial inhibition functions of the surface of the medical material at the same time, a micro-nano structure is prepared on the surface of the medical material after the coating or the plating is prepared.

The micro-nano structure is formed by superposing structures with three levels of sizes, wherein a first-level structure is a micron-level groove structure, a second-level structure is a submicron-level stripe structure or an array protrusion structure, and a third-level structure is a nano-level protrusion structure. The first-level groove structure is composed of a groove with a width of 20 µm to 500 µm and a depth of 0.5 µm to 10 µm; and the grooves may be arranged in parallel or crosswise, and a spacing between two adjacent grooves is 0 to 500 µm. The second-level structure may be composed of a parallelly arranged stripe with a width of 100 nm to 1,000 nm, a height of 100 nm to 300 nm and a spacing of 100 nm to 1,000 nm, or an array protrusion with a bottom surface size of 50 nm to 500 nm and a height of 20 nm to 500 nm, and the second-level structure is distributed on a surface of the first-level structure. The third-level structure may be a submicron-level protrusion structure, or is composed of a structure of a nanoscale, such as a nano particle, a nano rod, a nano cone, a nano mesh, a nano sheet and a nano tube with a size of 1 nm to 200 nm, and the third-level structure is distributed on a surface of the second-level structure.

The micro-nano structure formed by superposing the multiple levels of sizes is prepared by one method or a combination of two or more methods of pulse laser machining, electrochemical surface processing, machining, NaOH hydrothermal method, sandblasting and acid etching, physical vapor deposition, chemical vapor deposition and nano imprinting.

The medical material comprises, but is not limited to, pure titanium and alloy thereof, nickel-titanium alloy, iron and alloy thereof, stainless steel, cobalt-chromium alloy, pure magnesium and alloy thereof, pure tantalum and alloy thereof, pure zinc and alloy thereof, copper alloy, pure gold, pure silver, pure platinum and other medical metal materials; alumina ceramic, zirconia ceramic, silicon nitride ceramic, a carbon material, hydroxyapatite, tricalcium phosphate and other medical ceramic materials; and polyethylene, polytetrafluoroethylene, polypropylene, polyether ether ketone and other medical polymer materials.

Due to a size effect, the first-level structure above effectively increases a surface area of the material, which can promote cell adhesion and growth. The second-level structure improves a roughness of a sample surface, and provides an adhesion site for cell adhesion, and meanwhile, these structures effectively reduce adhesion of a gram-positive bacterium and a gram-negative bacterium represented by Escherichia coli and Staphylococcus aureus, thus exerting a bacteriostatic action. The third-level structure may kill bacteria, without affecting cell growth. A combined effect of the three structures above makes the surface of the medical material have dual functions of promoting cell growth and inhibiting bacterial proliferation.

The present invention has the advantages and the beneficial effects as follows:

according to the present invention, by carrying out modification on the surface of the medical material and preparing at least one of the plating and the coating on the surface, the medical material is endowed with dual functions of promoting cell growth and inhibiting bacterial adhesion, the long-term effectiveness and reliability of surface function are ensured at the same time, and the problem that a surface of an existing medical device lacks biological activity and bacteriostatic performance can be solved.

### DESCRIPTION OF THE DRAWINGS

FIG. 1 shows three kinds of three-level micro-nano structures prepared by one step through a pulse laser machining method by using different processes in Embodiment 1, wherein (a), (b) and (c) are first-level, second-level and third-level structures of the micro-nano composite structure respectively;

FIG. 2 is a comparative diagram of an osteoblast, an endothelial cell and a smooth muscle cell under four surface adhesion conditions in Comparative Example 1;

FIG. 3 is a comparative diagram of Escherichia coli and Staphylococcus aureus under four surface adhesion conditions in Comparative Example 1;

FIG. 4 is a comparative diagram of adhesion conditions of the osteoblast on surfaces with different micro-nano structures;

FIG. 5 is a comparative diagram of adhesion conditions of the Escherichia coli on surfaces with different micro-nano structures;

FIG. 6 is a composition diagram of two surfaces in Embodiment 2;

FIG. 7 is a comparative diagram of adhesion conditions of the osteoblast on three surfaces in Comparative Example 3;

FIG. 8 is a comparative diagram of adhesion conditions of the Staphylococcus aureus on three surfaces in Comparative Example 3;

FIG. 9 is an XPS high-resolution spectrogram of a Ti element of a surface in Embodiment 3;

FIG. 10 is a comparative diagram of adhesion conditions of the endothelial cell on two surfaces in Comparative Example 4;

FIG. 11 is a comparative diagram of Ni ion dissolution amounts of two surfaces in Comparative Example 4; and

FIG. 12 is a composition analysis diagram of a surface in Embodiment 6.

### DETAILED DESCRIPTION OF PREFERRED EMBODIMENTS

Specific description is made hereinafter with reference to embodiments of preparing surfaces with cell promotion and bacterial inhibition functions for different medical materials. A coating and a plating on the surface may be prepared by a medical material surface modification method such as plasma injection, plasma sputtering coating, plasma spraying, laser cladding, pulse laser deposition, laser alloying, sol-gel method, electrochemical deposition, electrophoretic deposition, anodic oxidation or micro-arc oxidation, and may also be prepared by a combination of two or more methods above. A three-level micro-nano composite structure may be prepared on the surface of the medical material by methods of pulse laser machining, electrochemical surface processing, machining, NaOH hydrothermal method, sandblasting and acid etching, physical vapor deposition, chemical vapor deposition and nano imprinting, and may also be prepared by a combination of two or more methods above.

### Embodiment 1

In this embodiment, a surface containing Ca and P for cell promotion and bacterial inhibition with a three-level micro-nano composite structure was prepared on a pure titanium surface by a plasma injection method combined with pulse laser surface processing. Specific steps were as follows.

(1) The pure titanium surface was mechanically polished first.

(2) 2×10¹⁵ ions/cm² of Ca and 1×10¹⁵ ions/cm² P were injected into the titanium surface respectively by the plasma injection method.

Ion injection formed an amorphous layer with a thickness of about 400 nm on the titanium surface, and the amorphous layer could effectively improve the biocompatibility of the titanium surface, effectively promote the adhesion and proliferation of an osteoblast, and accelerate the integration of titanium with a bone tissue. Meanwhile, the amorphous layer could effectively improve the wear resistance and corrosion resistance of the titanium surface, and improve the long-term reliability of an implanted device.

(3) A sample was fixed on a machining platform, a laser machining process was adjusted, and the whole surface was scanned. Parameters of the used pulse laser and ranges of the machining process were: a wavelength of 800 nm, a frequency of 1 kHz, a pulse width of 140 fs, average power of 5 mW to 1,000 mW, a machining speed of 0.1 mm/s to 10 mm/s, a light spot diameter of about 50 µm to 200 µm, and a laser scanning line spacing of 50 µm to 200 µm.

The following process was selected to prepare the three-level composite structure on the titanium surface above.

First machining was carried out according to power of 100 mW, a frequency of 1 kHz, a pulse width of 140 fs, a machining speed of 1 mm/s, a light spot diameter of about 100 µm, and a laser scanning line spacing of 80 µm. Second machining was carried out according to power of 20 mW, a frequency of 1 kHz, a pulse width of 140 fs, a machining speed of 1 mm/s, a light spot diameter of about 100 µm, and a laser scanning line spacing of 80 µm.

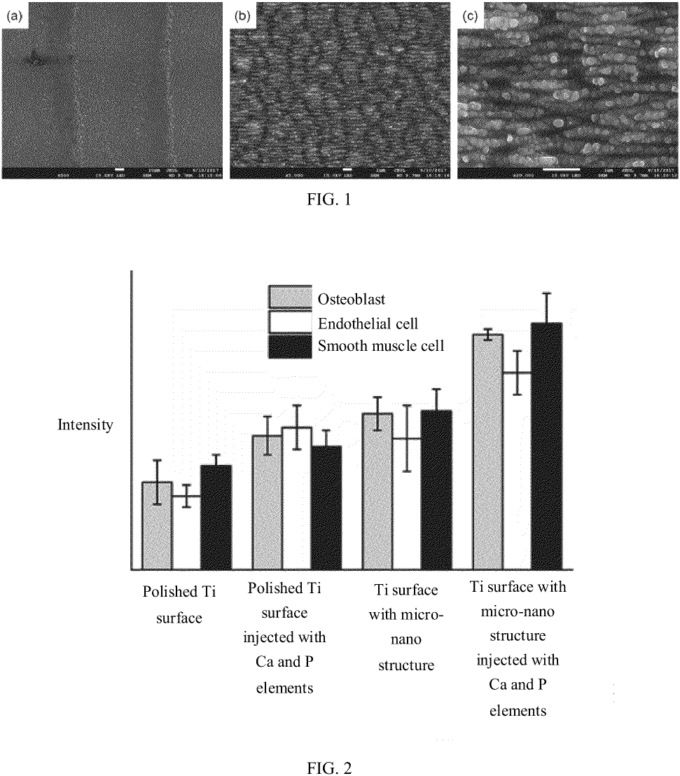

As shown in FIG. 1, the micro-nano composite structure prepared by the two machining operations was a three-level composite structure, and the structure comprised a first-level structure with a width of about 90 µm; a second-level structure which was a stripe with a width of 150 nm to 300 nm, wherein a spacing between adjacent stripes was 10 nm to 50 nm; and a third-level structure which was a nano particle of 20 nm to 300 nm distributed non-uniformly.

A part of material on a top layer of the titanium surface was removed during pulse laser machining, and since a thickness of a machining-affected layer was less than that of the amorphous layer of Ca and P, Ca and P remained on the titanium surface. The three-level micro-nano composite structure could further improve cell promotion and bacterial inhibition abilities of the titanium surface, make a physical structure exist stably, and ensure the long-term reliability of cell promotion and bacterial inhibition functions of the titanium surface.

### Comparative Example 1

In this comparative example, influences of a polished titanium surface, a polished titanium surface injected with Ca and P, a titanium surface with a three-level composite structure, and a titanium surface containing Ca and P and having the three-level composite structure on cell and bacterial adhesion were compared.

The polished titanium surface was prepared by a mechanical polishing method. A preparation method of the polished titanium surface injected with Ca and P was that: the polished titanium surface was prepared by mechanical polishing first, and 2×10¹⁵ ions/cm² of Ca and 1×10¹⁵ ions/cm² of P were injected into the surface respectively by the plasma injection method subsequently. A preparation method of the titanium surface with the three-level micro-nano structure was that: the titanium surface was polished by the mechanical polishing method, and the surface was subjected to laser machining subsequently. The laser machining process was the laser machining process used in Embodiment 1. A preparation method of the titanium surface containing Ca and P and having the three-level composite structure was that: the titanium surface was polished by the mechanical polishing method first, 2×10¹⁵ ions/cm² of Ca and 1×10¹⁵ ions/cm² of P were injected into the surface respectively by the plasma injection method, and the micro-nano composite structure was prepared on the surface by the laser machining method subsequently.

A cell adhesion experiment was carried out first, an osteoblast, an endothelial cell and a smooth muscle cell were inoculated on four sample surfaces respectively, and adhesion conditions of various cells on various surfaces were detected 1 day after inoculation. An experimental method was that: 40 µl of 5 × 10⁴/ml cell suspension was dropwise added on the four sample surfaces respectively, and cultured for 24 hours respectively, then the surfaces were washed with PBS, and numbers of cells adhered to the sample surfaces were compared by a CCK-8 method. As shown in FIG. 2, 1 day after inoculation, the three cells were all adhered to the four surfaces, wherein numbers of living cells on the titanium surface injected with Ca and P and the titanium surface with the micro-nano composite structure were higher than that on the polished titanium surface, which indicated that the amorphous layer prepared on the titanium surface by plasma injection improved the cell compatibility of the titanium surface, and the three-level micro-nano composite structure also promoted the cell adhesion. A number of cells on the titanium surface containing Ca and P and having the micro-nano composite structure was the highest, which indicated that a combined effect of Ca and P injection and micro-nano composite structure was more obvious than that of single Ca and P injection or micro-nano composite structure in promoting cell adhesion.

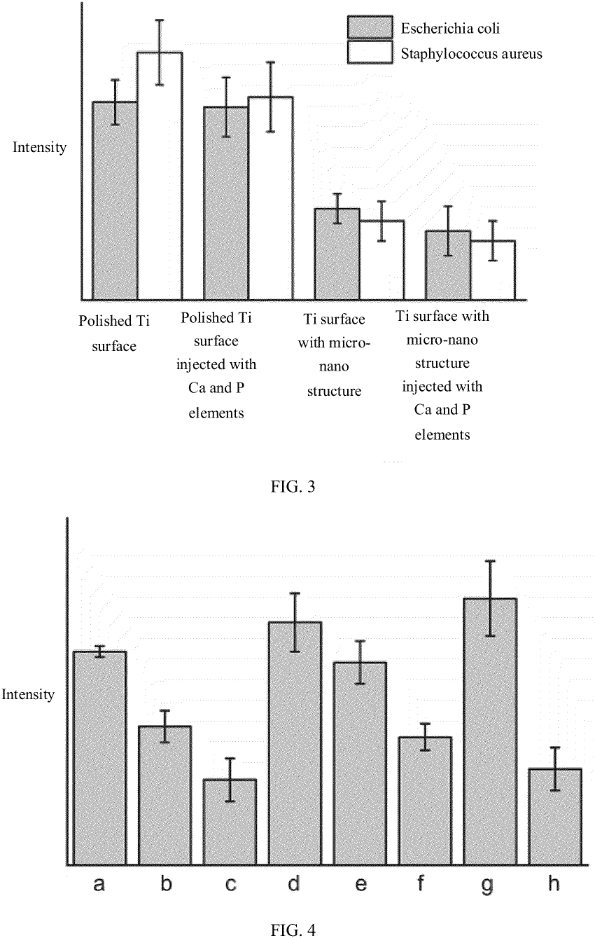

A bacterial adhesion experiment was carried out subsequently, and an experimental method was that: 40 µl of 10⁷/ml Escherichia coli liquid and 40 µl of 10⁷/ml Staphylococcus aureus liquid were dropwise added on four sample surfaces respectively, and cultured for 6 hours, then the surfaces were washed with PBS, and bacteria not adhered to the sample surfaces were removed. The surfaces were subjected to fluorescence staining, and observed by a laser confocal microscope, and fluorescence intensities of any 10 positions on each surface were counted. As shown in FIG. 3, 6 hours after inoculation, two bacteria were both adhered to the four surfaces, and fluorescence intensities of the two surfaces with the micro-nano composite structure were significantly lower than those of the two polished surfaces, which indicated that numbers of bacteria adhered to the two surfaces were significantly lower. This result proved that the surface with the micro-nano composite structure had an obvious effect of inhibiting adhesion of a gram-negative bacterium and a gram-positive bacterium.

### Comparative Example 2

In this comparative example, influences of different micro-nano composite structures on bacterial and cell adhesion were studied. Seven surfaces of a to h were prepared on a titanium surface, and specific preparation methods and surface structures were as follows.

Sample a: a micron-groove, which was namely a first-level structure, was prepared on the titanium surface by a mechanical machining method, with a width of about 90 µm and a depth of about 1 µm.

Sample b: a stripe structure, which was namely a second-level structure, was prepared on the titanium surface by a pulse laser machining method, wherein parameters of the used pulse laser and the machining process were: a wavelength of 800 nm, a frequency of 1 kHz, a pulse width of 140 fs, power of 40mW, a machining speed of 1 mm/s, a light spot diameter of about 100 µm, and a laser scanning line spacing of 100 µm. A width of the stripe of the second-level structure prepared was 150 nm to 300 nm, a spacing between adjacent stripes was 20 nm to 50 nm, a surface of the stripe was smooth, and there were almost no nano structures on the surface.

Sample c: a nano particle, which was namely a third-level structure, was prepared on the titanium surface by a pulse laser remelting method, wherein the used laser and the machining process were: a wavelength of 1,030 nm, a frequency of 50 MHz, a pulse width of 150 fs, power of 10 W, a machining speed of 10 mm/s, a light spot diameter of about 200 µm, and a laser scanning line spacing of 180 µm. The obtained structure was a nano particle of 20 nm to 300 nm distributed densely.

Sample d: a micron-groove was prepared on the titanium surface by machining first, with a width of about 80 µm and a depth of about 1 µm, and the whole surface was scanned by a pulse laser subsequently, wherein parameters of the used pulse laser and the machining process were: a wavelength of 800 nm, a frequency of 1 kHz, a pulse width of 140 fs, power of 40 mW, a machining speed of 1 mm/s, a light spot diameter of about 100 µm, and a laser scanning line spacing of 100 µm. The stripe structure, which was namely the second-level structure, with a width of 150 nm to 300 nm and a spacing of 20 nm to 50 nm, was prepared on a surface of the first-level structure, a surface of the stripe was smooth, and there were almost no nano structures on the surface.

Sample e: a micron-groove was prepared on the titanium surface by machining first, with a width of about 80 µm and a depth of about 1 µm, and the whole surface was scanned by a pulse laser subsequently, wherein parameters of the used pulse laser and the machining process were: a wavelength of 1,030 nm, a frequency of 50 MHz, a pulse width of 150 fs, power of 10 W, a machining speed of 10 mm/s, a light spot diameter of about 200 µm, and a laser scanning line spacing of 180 µm. A nano particle of 40 nm to 300 nm distributed densely was prepared on the surface of the first-level structure.

Sample f: the second-level stripe structure was prepared on the titanium surface by the pulse laser machining method first, wherein parameters of the used pulse laser and the machining process were: a wavelength of 800 nm, a frequency of 1 kHz, a pulse width of 140 fs, power of 40 mW, a machining speed of 1 mm/s, a light spot diameter of about 100 µm, and a laser scanning line spacing of 100 µm. A width of the stripe of the second-level structure prepared was 150 nm to 300 nm, a spacing between adjacent stripes was 20 nm to 50 nm, a surface of the stripe was smooth, and there were almost no nano structures on the surface; and second scanning was carried out by the same laser subsequently according to a laser wavelength of 800 nm, a frequency of 1 kHz, a pulse width of 140 fs, power of 20 mW, a machining speed of 1 mm/s, a light spot diameter of about 100 µm and a laser scanning line spacing of 100 µm.

Sample g: the titanium surface with the three-level micro-nano composite structure was prepared by the same method and process as those in Embodiment 1.

Sample h: the polished titanium surface was prepared by the mechanical polishing method.

A cell adhesion experiment and a bacterial adhesion experiment were carried out on the eight sample surfaces.

A specific method of the cell experiment was as follows. 40 µl of 5 × 10⁴/ml osteoblast suspension was dropwise added on the eight sample surfaces, and cultured for 24 hours, then the surfaces were washed with PBS, and numbers of cells adhered to the sample surfaces were compared by a CCK-8 method.

OD values of the samples measured by the CCK8 method were directly proportional to the numbers of cells adhered to the sample surfaces, and the surface with the higher fluorescence intensity had a more obvious effect of promoting cell adhesion. As shown in FIG. 4, effects of promoting cell adhesion of different surfaces were sorted as follows: d, g > a, e > b, f > c, h. This result showed that structures of different surfaces had different effects of promoting cell adhesion when the material composition was the same. The surface d and the surface g had the most obvious effect of promoting adhesion of the three cells, the surface c and the surface h had the least effect of promoting cell adhesion, the surface a and the surface e had similar effects of promoting cell adhesion, and the surface b and the surface f had similar effects. This result proved that the first-level and second-level structures had the effect of promoting cell adhesion, wherein the first-level structure increased a specific surface area of the material and increased an adherable area of the cells, the second-level structure increased a surface roughness and provided more adhesion sites for cell adhesion, and the combination of the first-level and second-level structures had the most obvious effect of cell adhesion; and the third-level structure had no obvious influence on cell adhesion.

A specific method of the bacterial adhesion experiment was that: 40 µl of 10⁷/ml Escherichia coli liquid was dropwise added on the eight sample surfaces respectively, and cultured for 6 hours, then the surfaces were washed with PBS, and bacteria not adhered to the sample surfaces were removed. The surfaces were subjected to fluorescence staining, and observed by a laser confocal microscope, and fluorescence intensities of any 10 positions on each surface were counted.

An average value of a fluorescence intensity of each sample surface was directly proportional to a number of bacteria adhering to the surface, and the lower the fluorescence intensity was, the stronger the effect of inhibiting bacterial adhesion of the surface was. As shown in FIG. 5, the eight surfaces had different effects of adhering Escherichia coli. Bacteria were adhered to the polished surface (surface h) in large quantity. Since the first-level structure of the surface a increased the specific surface area, an adherable area of the bacteria was larger, and an adhesion capacity of the bacteria was larger. Compared with the surfaces a and h, other surfaces all had the effect of inhibiting bacterial adhesion. This result showed that the second-level and third-level structures had the effect of inhibiting Escherichia coli, the second-level structure effectively reduced an adhering area of the bacteria, and the third-level structure further reduced the adherable area of the bacteria. Meanwhile, the third-level nano structure could pierce cell membranes of the bacteria to kill the bacteria. The second-level and third-level composite structures had the most obvious effect of inhibiting adhesion of the two bacteria.

This result proved an influence of a multi-level micro-nano composite structure on adhesion of the cells and the bacteria on the structure. The first-level structure above effectively increased the surface area of the material, which could promote cell adhesion and growth. The second-level structure improved the roughness of the sample surface, and provided the adhesion site for cell adhesion, and meanwhile, these structures effectively reduced bacterial adhesion, thus exerting a bacteriostatic action. The third-level structure could kill the bacteria, without affecting cell growth.

### Embodiment 2

316L stainless steel was widely used medical alloy, but this material still had the problems of lacking surface activity and having no bacteriostatic ability. In this embodiment, a Ti-hydroxyapatite coating was prepared on a 316L stainless steel surface by magnetron sputtering and plasma spraying in sequence, a micro-nano composite structure was prepared on the surface by a femtosecond laser machining method subsequently, and a bioactive coating with good corrosion resistance and high bonding strength was prepared on the 316L surface. Specific steps were as follows.

(1) The 316L stainless steel surface was polished by a mechanical polishing method.

(2) Pure titanium was sputtered on the surface by magnetron sputtering to form a dense titanium transition layer on the surface.

(3) Molten hydroxyapatite powder was sprayed on the surface by a plasma spraying method to form a hydroxyapatite ceramic layer on the surface.

(4) The hydroxyapatite coating prepared by the plasma spraying method had the problems of insufficient bonding strength and no bacteriostatic ability, so that the surface was processed by the pulse laser machining method. Parameters of the used pulse laser and the machining process were: a wavelength of 1030 nm, a frequency of 320 kHz, a pulse width of 150 fs, power of 8 W, a machining speed of 100 mm/s, a light spot diameter of about 50 µm, and a laser scanning line spacing of 40 µm. A multi-level micro-nano composite structure was obtained on the surface, wherein a first-level structure was a groove structure with a width of about 35 µm, a second-level structure was a structure with a width of about 150 nm to 300 nm, wherein a spacing between adjacent stripes was 20 nm to 50 nm, and a third-level structure was a nano particle with a diameter of 200 nm to 400 nm distributed on a surface of the second-level structure.

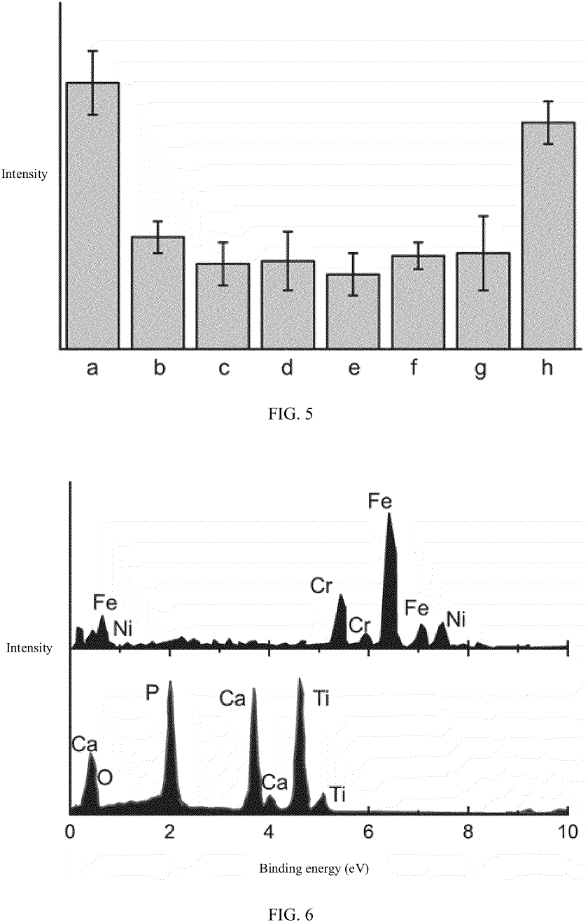

FIG. 6 showed EDS spectra of a polished 316L stainless steel surface and a 316L stainless steel surface provided with a titanium-hydroxyapatite coating of a micro-nano structure. Compared with the polished 316L stainless steel surface, the 316L stainless steel surface provided with the titanium-hydroxyapatite coating of the micro-nano structure contained Ti, Ca, P and other elements.

### Comparative Example 3

In this comparative example, an osteoblast and Staphylococcus aureus were inoculated on a smooth 316L stainless steel surface, a 316L stainless steel surface with a smooth titanium-hydroxyapatite coating and a 316L stainless steel surface with a titanium-hydroxyapatite coating of a micro-nano composite structure respectively, and influences of the three surfaces on the adhesion and growth of the osteoblast and the adhesion and proliferation of the Staphylococcus aureus were compared.

The smooth 316L stainless steel surface above was prepared by a mechanical polishing method, the 316L stainless steel surface with the titanium-hydroxyapatite coating was prepared by magnetron sputtering combined with plasma spraying in Embodiment 2, and the 316L stainless steel surface with the titanium-hydroxyapatite coating of the micro-nano composite structure was prepared by magnetron sputtering, plasma spraying and laser machining in Embodiment 2.

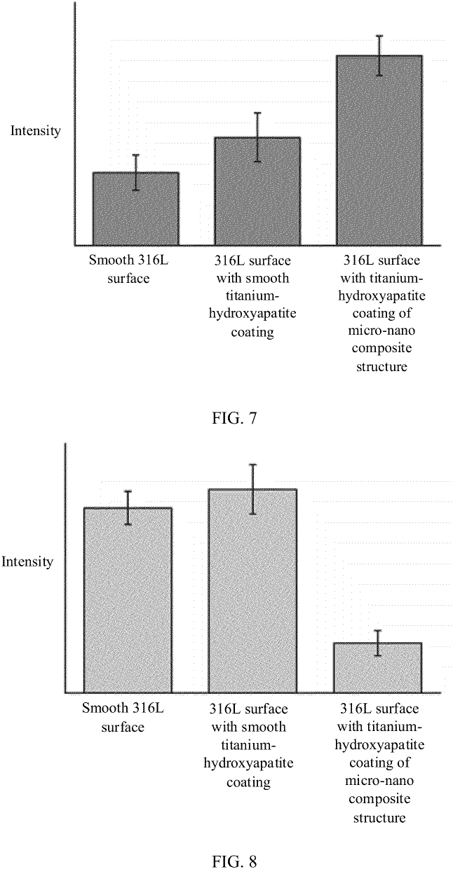

An osteoblast proliferation experiment was carried out on the three surfaces first, and an experimental method was that: 40 µl of 5 × 10⁴/ml osteoblast suspension was dropwise added on the three sample surfaces, and cultured for 24 hours, then the surfaces were washed with PBS, and numbers of cells adhered to the sample surfaces were compared by a CCK-8 method. As shown in FIG. 7, numbers of osteoblasts adhered to the three surfaces were sorted from large to small as follows: the 316L stainless steel surface with the titanium-hydroxyapatite coating of the micro-nano composite structure, the 316L stainless steel surface with the smooth titanium-hydroxyapatite coating, and the smooth 316L stainless steel surface. The 316L stainless steel surface was a biologically inert surface, and the titanium-hydroxyapatite coating effectively improved the biological activity of the 316L stainless steel surface and improved the cell compatibility, thus promoting the adhesion of the osteoblast. The micro-nano composite structure was prepared on the 316L stainless steel surface with the smooth titanium-hydroxyapatite coating by a femtosecond laser machining method, which further improved the biological activity of the surface, thus promoting the adhesion and proliferation of the osteoblast on the surface.

A Staphylococcus aureus adhesion experiment was carried out subsequently, and a specific method was that: 40 µl of 10⁷/ml Staphylococcus aureus liquid was dropwise added on the three sample surfaces, and cultured for 6 hours, then the surfaces were washed with PBS, and bacteria not adhered to the sample surfaces were removed. The surfaces were subjected to fluorescence staining, and observed by a laser confocal microscope, and fluorescence intensities of any 10 positions on each surface were counted. As shown in FIG. 8, in the three surfaces, the two smooth surfaces had the largest number of Staphylococcus aureus adhered, and the surface with the micro-nano composite structure had a significantly reduced number of Staphylococcus aureus adhered, which proved that the micro-nano composite structure had an effect of inhibiting the adhesion of Staphylococcus aureus.

In this comparative example, the experiment proved that the 316L stainless steel surface with the titanium-hydroxyapatite coating of the micro-nano composite structure prepared by the method in Embodiment 2 could inhibit the adhesion of Staphylococcus aureus while promoting cell adhesion and growth.

### Embodiment 3

In this embodiment, a TiO₂ coating with a micro-nano composite structure was prepared on a TC4 surface by machining combined with hydrothermal processing with a NaOH solution. Specific steps were as follows.

(1) The surface was mechanically polished first.

(2) A first-level groove structure was prepared on the surface by using a precision five-axis machining center.

(3) A second-level groove structure was prepared on an inner surface of the groove prepared in the step (2) by using the precision five-axis machining center.

(4) The surface with the first-level and second-level composite structures was soaked in the NaOH solution and put in a high-pressure reaction kettle, and a layer of third-level protrusion structure in a nano rod shape grew on the surface by the hydrothermal method. A concentration of the NaOH solution used in the hydrothermal reaction was 1 mol/L to 2 mol/L, the reaction lasted for 3 hours to 10 hours, and the reaction was carried out at a temperature of 120° C. to 240° C.

The surface with the three-level micro-nano structure was obtained by machining, wherein the first-level structure had a width of about 400 µm and a depth of about 5 µm; the second-level stripe structure was distributed on a surface of the first-level structure, wherein the stripe had a width of about 300 nm and a height of about 200 nm, and a spacing between two adjacent stripes was about 200 nm; and the third-level structure was a protrusion in a nano rod shape with a diameter of about 40 nm to 80 nm, and the third-level structure was uniformly distributed on a surface of the second-level stripe structure. FIG. 9 showed analysis results of surface composition by an X-ray photoelectron spectrum. A high-resolution analysis spectrum of a Ti element was analyzed, and results showed that TiO₂ was formed on the surface.

### Embodiment 4

Superelasticity and shape memory effects caused by NiTi alloy were widely used to prepare various inner supports, but a nickel-titanium alloy surface also had the problems of insufficient biological activity and no bacteriostatic ability, and meanwhile, a NiTi alloy device also had the problem of biological toxicity caused by Ni ion dissolution. In this embodiment, a surface with a C-containing coating of a micro-nano composite structure was prepared on the NiTi alloy surface by a micro-arc oxidation method combined with a hydrothermal method and an ion injection method. Specific steps were as follows.

(1) The surface to be processed was mechanically polished first.

(2) The sample was processed by micro-arc oxidation in an ethylene glycol electrolyte. A micro pit with a diameter of 1 µm to 10 µm and a depth of 100 nm to 500 nm was formed on the surface.

(3) The sample was placed in a NaOH solution for hydrothermal processing, and a layer of protrusion structure in a rod shape with a diameter of about 30 nm grew on the surface with the micro pit.

(4) The sample was placed in a C₂H₂ atmosphere, and the C element was injected into the surface by a PIII method.

The C-containing coating of the micro-nano composite structure was prepared on the NiTi alloy surface by the method above.

### Comparative Example 4

In this comparative example, influences of NiTi alloy surfaces with three-level micro-nano structures injected with and not injected with a C element on adhesion and proliferation of an endothelial cell were compared first, and dissolution capacities of Ni ions of the two surfaces in a simulated body fluid were detected subsequently.

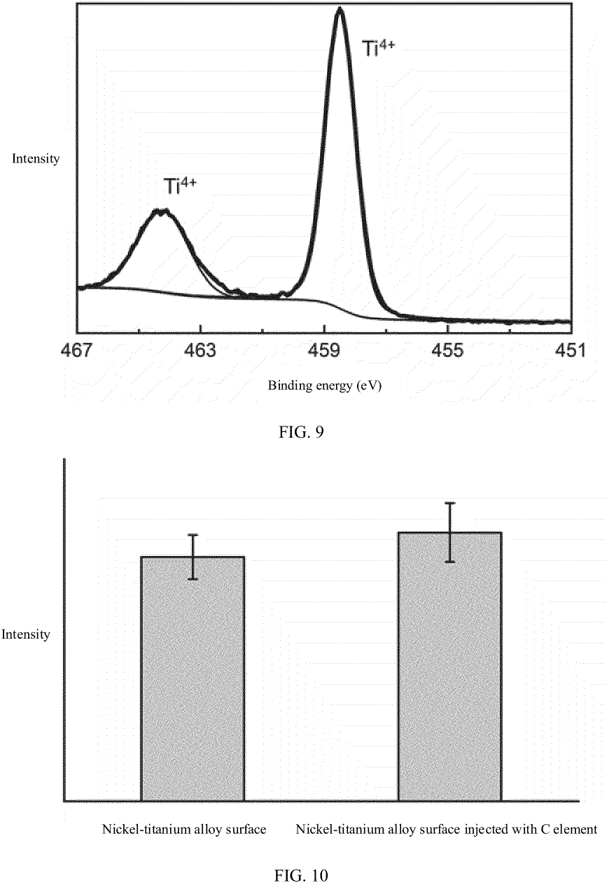

An endothelial cell proliferation experiment was carried out on the two surfaces first, and an experimental method was that: 40 µl of 5 × 10⁴/ml endothelial cell suspension was dropwise added on the two sample surfaces, and cultured for 24 hours, then the surfaces were washed with PBS, and numbers of cells adhered to the sample surfaces were compared by a CCK-8 method. As shown in FIG. 10, adhesion capacities of endothelial cells on the NiTi alloy surface with the micro-nano composite structure injected with the C element and the NiTi alloy surface with the micro-nano composite structure not injected with the C element were basically consistent, so that the injection of the C element did not affect the biocompatibility of the surface.

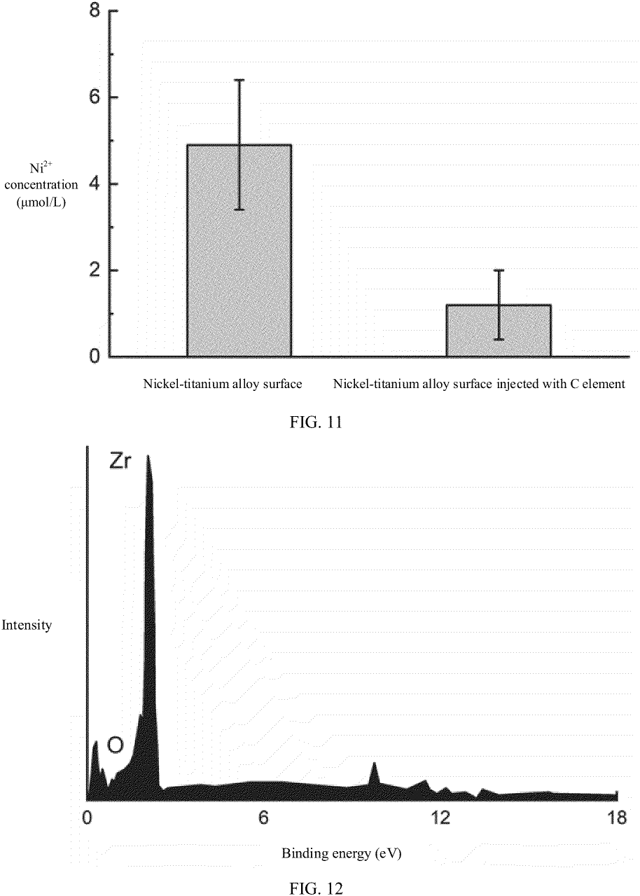

The dissolution capacities of Ni ions of the two surfaces in the simulated body fluid were detected by inductively coupled plasma mass spectrometry subsequently. A specific method was that: the two surfaces were respectively soaked in 1-fold simulated body fluid at a constant temperature of 37° C. for 24 hours, and concentrations of Ni ions in the two leaching solutions were detected by an ICP-MS method. As shown in FIG. 11, the dissolution capacity of Ni ions of the surface not injected with the C element was obviously higher than that of the surface injected with the C element.

This result showed that the injection of the C element could effectively reduce the dissolution of Ni ions without affecting the compatibility of the endothelial cell of the NiTi alloy surface.

### Embodiment 5

In this embodiment, a TiO₂ coating with a three-level composite micro-nano structure was prepared on a Ti surface by a machining method combined with an anodic oxidation method. Specific steps were as follows.

(1) The Ti surface to be processed was mechanically polished first.

(2) A first-level groove structure was prepared on the surface by using a precision five-axis machining center.

(3) A second-level groove structure was prepared on an inner surface of the groove prepared in the step (2) by using the precision five-axis machining center.

(4) A third-level nano tube structure was prepared on the surfaces of the first-level and second-level structures by the anodic oxidation method, and meanwhile a dense TiO₂ layer was formed on the surface. The anodic oxidation process was that: 1% HF solution was used as an electrolyte, an anodic oxidation processing voltage was 30 V, and the processing lasted for 15 minutes.

The surface with the three-level micro-nano structure was obtained by machining, wherein the first-level structure had a width of about 40 µm and a depth of about 6 µm; the second-level stripe structure was distributed on the surface of the first-level structure, wherein the stripe had a width of about 300 nm and a height of about 200 nm, and a spacing between two adjacent stripes was about 200 nm; and the third-level structure was a nano tube with a diameter of about 10 nm to 20 nm, and the third-level structure was uniformly distributed on the surface of the second-level stripe structure.

### Embodiment 6

In this embodiment, a ZrO₂ coating with a three-level micro-nano structure was prepared on a polyether ether ketone surface by a sol-gel method and a pulse laser machining method.

The ZrO₂ coating with a thickness of about 500 nm was prepared on the polyether ether ketone surface by the sol-gel method first. As shown in FG. 12, a layer of dense ZrO₂ coating was formed on the surface.

The surface was processed by a pulse laser subsequently, wherein parameters of laser machining were: a wavelength of 800 nm (a frequency of 1 kHz, a pulse width of 200 fs), average power of 30 mW, a machining speed of 1 mm/s, a light spot diameter of about 500 µm, and a laser scanning line spacing of 400 µm. The surface with the three-level micro-nano structure was obtained by machining, wherein the first-level structure had a width of 450 µm and a depth of 300 nm; the second-level stripe structure was distributed on a surface of the first-level structure, wherein the stripe had a width of about 250 nm and a height of about 100 nm, and a spacing between two adjacent stripes was about 250 nm; and the third-level structure was a nano protrusion with a diameter of about 50 nm to 100 nm, and the third-level structure was non-uniformly distributed on a surface of the second-level stripe structure.

The above are only the preferred embodiments of the present invention, and it should be pointed out that those of ordinary skills in the art may further make several modifications and improvements without departing from the concept of the present invention, and these modifications and improvements all fall within the scope of protection of the present invention.

We claim:

1. A medical material, wherein a structure for promoting cell growth and inhibiting bacterial adhesion is prepared on a surface of the material, the structure is a micro-nano structure formed by superposing multiple levels of sizes, the micro-nano structure formed by superposing the multiple levels of sizes comprises a first-level structure which is a micron-level groove structure, a second-level structure which is a submicron-level stripe structure or an array protrusion structure and a third-level structure which is a nano-level protrusion structure, the second-level structure is distributed on a surface of the first-level structure, and the third-level structure is distributed on a surface of the second-level structure;
the groove of the first-level structure has a width of 20 µm to 500 µm and a depth of 0.5 µm to 10 µm;
the submicron-level stripe of the second-level structure has a width of 100 nm to 1,000 nm and a height of 100 nm to 300 nm; and the array protrusion has a height of 20 nm to 500 nm;
the nano-level protrusion of the third-level structure is one form or a combination of two or more forms of nano particle, nano rod, nano cone, nano mesh, nano sheet and nano tube of 1 nm to 200 nm; and
a machining method of the medical material comprises:
carrying out modification on a surface of the medical material first; and
preparing a micro-nano structure formed by superposing multiple levels of sizes subsequently;
wherein the modification refers to preparing at least one of a coating and a plating on the surface of the medical material, and the plating comprises one or two or more elements of Ca, Zn, Fe, Ta, Mo, Ti, Au, Pt, Cu, Ag, P, Se, B, C, N, Ar and He; and the coating comprises one or two or more compounds of hydroxyapatite, TiO₂, SiO₂ and ZrO₂, a thickness of at least one of the coating and the plating is 10 nm to 500 µm, and a height difference between a highest point and a lowest point of the micro-nano structure formed by superposing the multiple levels of sizes is less than the thickness of at least one of the coating and the plating.

2. The medical material according to claim 1, wherein at least one of the coating and the plating is prepared by one method or a combination of two or more methods of plasma injection, plasma sputtering coating, plasma spraying, laser cladding, pulse laser deposition, laser alloying, sol-gel method, electrochemical deposition, electrophoretic deposition, anodic oxidation or micro-arc oxidation.

3. The medical material according to claim 1, wherein the micro-nano structure formed by superposing the multiple levels of sizes is prepared by one method or a combination of two or more methods of pulse laser machining, electrochemical surface processing, machining, NaOH hydrothermal method, sandblasting and acid etching, physical vapor deposition, chemical vapor deposition and nano imprinting.

4. The medical material according to claim 1, wherein the medical material is a medical metal material, a medical ceramic material or a medical polymer material.
